(19) United States
(12) Patent Application Publication
(10) Pub. No.: US 2002/0103426 A1
(43) Pub. Date: Aug. 1, 2002

(54) Electrophysiology catheter

(76) Inventors: Garland L. Segner, Watertown, MN (US); Roger N. Hastings, Maple Grove, MN (US)

Correspondence Address: Bryan K. Wheelock, Harness, Dickey & Pierce, P.L.C., 7700 Bonhomme, Suite 400, St. Louis, MO, 63105, US

(21) Appl. No.: 09/771,954

(22) Filed: Jan. 29, 2001

(51) Int. Cl. A61B 5/042; A61B 18/14; A61N 1/05

(52) U.S. Cl. 600/374; 606/41; 607/122

(57) ABSTRACT

An electrophysiology catheter has a proximal end and a distal end. There is a first generally hollow electrode member at the distal end, having a generally cylindrical sidewall and a dome shaped distal end. There is a magnet member at least partially within the hollow electrode member. The magnet member may be a permanent magnet or a permeable magnet material. The magnet is sufficient size and strength to align the distal end of the electrophysiology catheter inside the body of a patient with an externally applied magnetic field.

### BACKGROUND OF THE INVENTION

This invention relates to electrophysiology catheters, and in particular to an electrophysiology catheter adapted for magnetic navigation.

Electrophysiology catheters are elongate medical devices that are introduced into the body and are used for sensing electrical properties of tissues in the body; applying electrical signals to the body for example for cardiac pacing; and applying energy to the tissue for ablation. Electrophysiology catheters have a proximal end, a distal end, and two or more electrodes on their distal end. Recently, electrophysiology catheters have been made with electrodes having openings in their distal ends for passage of normal saline solution which cools the surface tissues to prevent blood clotting. These electrodes can be difficult to navigate into optimal contact with the tissues using conventional mechanical pull wires.

### SUMMARY OF THE INVENTION

The electrophysiology catheter of this invention is particularly adapted for magnetic navigation. Generally, the catheter has a proximal end and a distal end and a first generally hollow electrode member at the distal end. The first electrode has a generally cylindrical sidewall and a dome shaped distal end. There is a second electrode spaced proximally from the first electrode, and in general there are multiple ring electrodes spaced at equal distances proximal to the first electrode. In accordance with the principles of this invention, there is a magnet member at least partially, and preferably substantially entirely, within the hollow electrode member. The magnet member can be a permanent magnet or a permeable magnet. The magnet member is sized and shaped so that it can orient the distal end of the catheter inside the body under the application of a magnetic field from an external source magnet. The magnet member is preferably responsive to a magnetic field of 0.1 T, and preferably less. The magnet member allows the distal end of the electrophysiology catheter to be oriented in a selected direction with the applied magnetic field, and advanced.

Because the magnet member is disposed in the hollow electrode, the distal end portion of the catheter remains flexible to facilitate orienting and moving the catheter within the body.

### BRIEF DESCRIPTION OF THE DRAWINGS

FIG. 1 is a longitudinal cross section of a catheter constructed according to the principles of this invention;

FIG. 2 is a longitudinal cross section of an alternate construction of a catheter constructed according to the principles of this invention, adapted to deliver irrigating fluid to the distal end; and

FIG. 3 is a is longitudinal cross sectional view of the alternate construction of a catheter constructed according to the principles of this invention, showing a separate line for providing irrigating fluid to the distal end.

Corresponding reference numerals indicate corresponding parts throughout the several views of the drawings.

### DETAILED DESCRIPTION OF THE INVENTION

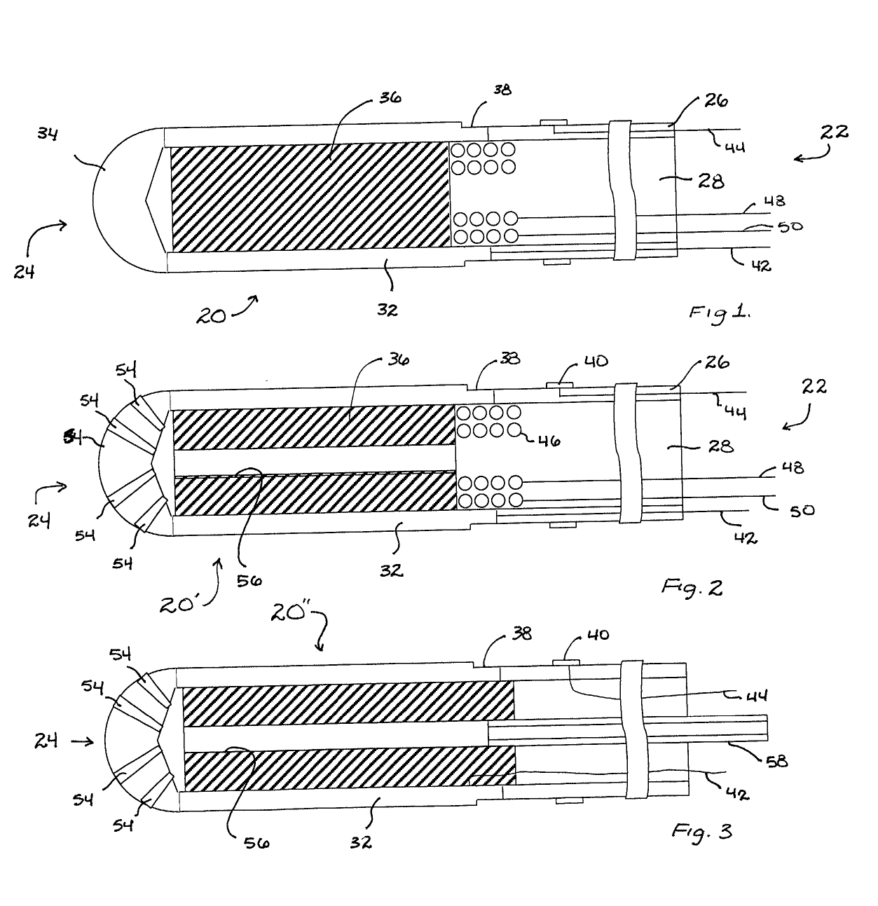

An electrophysiology catheter constructed according to the principles of this invention is indicated generally as 20 in FIG. 1. The electrophysiology catheter 20 has a proximal end 22 and a distal end 24. The catheter 20 is preferably a hollow flexible tubular member comprising a sidewall 26 with a lumen 28 therethrough. The catheter 20 can be made from Pebax™.

The electrophysiology catheter 20 of the present invention has a first generally hollow electrode member 30 on its distal end. The electrode member 30 has a generally cylindrical sidewall 22 and blunt, rounded dome-shaped 24. In the preferred embodiment, the electrode member 30 is preferably about 0.250 inches long, and has an external diameter of about 0.1044 inches. According to the principles of this invention, the electrode member 30 is hollow, opening to the proximal end. In the preferred embodiment the electrode member has a cavity that is about 0.205 to about 0.210 inches long, with a diameter of between about 0.091 and 0.095 inches. A magnet member 36 is disposed substantially entirely within the electrode member 30. The magnet member 36 is preferably a solid cylindrical mass of a permanent magnetic material, such as Neodymium-Iron-Boron (Nd—Fe—B) or Samarium-Cobalt, or a permeable magnetic material, such as hiperco.

The distal end portion 30 of the electrode 30 has a recessed diameter, facilitating joining the electrode 28 to the tube forming the catheter. In the preferred embodiment this recessed distal end portion 38 is about 0.05 inches long, and has an outside diameter of about 0.103 inches.

In an alternate construction of the preferred embodiment indicated generally as 20′ in FIGS. 2 and 3, there are a plurality of openings 40 in the dome 30, and there is at least one passage through the magnet member 36, such as passage 42 extending axially through the center of the magnet member, for the passage of irrigation fluid. The fluid can be provided through the lumen 28 of the catheter as shown in FIG. 2, or a separate line 44 can be provided to provide irrigating fluid to the distal end of the electrode as shown in FIG. 3.

A second annular electrode 46 is positioned on the exterior sidewall 26 of the catheter 20, spaced proximally from the first electrode member 30. Lead wires 48 and 50 extend proximally from the electrodes 28 and 40. These lead wires can pass through the lumen 28 of the catheter (as shown in FIG. 3), or they can be embedded in the sidewall 26 (as shown in FIG. 2). The proximal ends of the lead wires 48 and 50 can be electrically connected to an apparatus for sensing the potential the tissue between the electrodes, or to a device for applying an electric charge to the tissue between the electrodes, or to a device for applying electrical energy to the tissue for ablation between the tip electrode and a grounding pad on the patient.

By providing the magnet inside the first electrode, the distal end of the catheter remains more flexible, making it easier to navigate.

What is claimed is:

1. An electrophysiology catheter having a proximal end and a distal end, a first generally hollow electrode member at the distal end, the first electrode having a generally cylindrical sidewall and a dome shaped distal end, and a second electrode spaced proximally from the first electrode, and a magnet member at least partially within the hollow electrode member.

2. The electrophysiology catheter according to claim 1 wherein the magnet member is a permanent magnet.

3. The electrophysiology catheter according to claim 1 wherein the magnet member is a permeable magnet material.

4. The electrophysiology catheter according to claim 1 wherein the magnet is sufficient size and strength to align the distal end of the electrophysiology catheter inside the body of a patient with an externally applied magnetic field.

5. The electrophysiology catheter according to claim 4 wherein the magnet member is a permanent magnet.

6. The electrophysiology catheter according to claim 4 wherein the magnet member is a permeable magnet material.

7. The electrophysiology catheter according to claim 1 wherein the magnet is sufficient size and strength to align the distal end of the electrophysiology catheter inside the body of a patient with an externally applied magnetic field of at least 0.1 T.

8. The electrophysiology catheter according to claim 7 wherein the magnet member is a permanent magnet.

9. The electrophysiology catheter according to claim 7 wherein the magnet member is a permeable magnet material.

10. The electrophysiology catheter according to claim 1 wherein the magnet member is substantially entirely within the hollow electrode member.

11. The electrophysiology catheter according to claim 1 wherein the first electrode has a plurality of openings in its distal end, and wherein the magnet has a passage therethrough for conducting fluid from the catheter to the distal end of the first electrode where it can exit the first electrode through the plurality of openings in the distal end.

12. The electrophysiology catheter according to claim 11 wherein the magnet member is a permanent magnet.

13. The electrophysiology catheter according to claim 11 wherein the magnet member is a permeable magnet material.

14. An improved electrophysiology catheter of the type having a generally hollow electrode member at its distal end, the first electrode member having a generally cylindrical sidewall and a dome shaped distal end, the improvement comprising a magnet member at least partly within the generally hollow electrode, the magnet of sufficient size and strength to align the first electrode inside a patient's body.

15. The electrophysiology catheter according to claim 14 wherein the magnet member is substantially entirely within the hollow electrode member.

16. The electrophysiology catheter according to claim 15 wherein the first electrode has a plurality of openings in its distal end, and wherein the magnet has a passage therethrough for conducting fluid from the catheter to the distal end of the first electrode where it can exit the first electrode through the plurality of openings in the distal end.

17. The electrophysiology catheter according to claim 15 wherein the magnet member is a permanent magnet.

18. The electrophysiology catheter according to claim 15 wherein the magnet member is a permeable magnet material.

19. An improved electrophysiology catheter of the type having a generally hollow electrode member at its distal end, the first electrode member having a generally cylindrical sidewall and a dome shaped distal end, the improvement comprising a magnet member at least partly within the generally hollow electrode, the magnet of sufficient size and strength to align the first electrode inside a patient's body with an externally applied magnetic field of at least about 0.1 T.

20. The electrophysiology catheter according to claim 19 wherein the first electrode has a plurality of openings in its distal end, and wherein the magnet has a passage therethrough for conducting fluid from the catheter to the distal end of the first electrode where it can exit the first electrode through the plurality of openings in the distal end.

21. The electrophysiology catheter according to claim 19 wherein the magnet member is substantially entirely within the hollow electrode member.

22. The electrophysiology catheter according to claim 21 wherein the magnet member is a permanent magnet.

23. The electrophysiology catheter according to claim 21 wherein the magnet member is a permeable magnet material.

24. A method of navigating an electrophysiology catheter of the type having a generally hollow electrode member at its distal end, the method comprising providing a magnet member at least partly within the hollow electrode member, and applying a magnetic field from a source magnet outside the body to the magnet member inside the hollow electrode member to orient the distal end of the electrophysiology catheter in a desired direction.

25. The method according to claim 24 wherein the magnet member is substantially entirely within the hollow electrode member.

26. The method according to claim 24 wherein the generally hollow electrode has a plurality of openings in its distal end, and wherein the magnet member has a passage therethrough for conducting fluid from the catheter to the distal end of the first electrode where it can exit the first electrode through the plurality of openings in the distal end, and further comprising the step of ejecting coolant through the openings in the electrode.
